(19) United States
(12) Patent Application Publication
(10) Pub. No.: US 2026/0013826 A1
(43) Pub. Date: Jan. 15, 2026

(54) ULTRASONIC SENSOR SYSTEM FOR CARDIOVASCULAR SYSTEM MONITORING

(71) Applicant: TDK U.S.A. Corporation, Uniondale, NY (US)

(72) Inventors: Alessandro Ovidio Paris RAMALLI, Prato (IT); Paolo MATTESINI, Subbiano (IT); Leonardo BALDASARRE, Varese (IT); Marco TRAVAGLIATI, Pavia (IT)

(73) Assignee: TDK U.S.A. Corporation, Uniondale, NY (US)

(21) Appl. No.: 19/268,890

(22) Filed: Jul. 14, 2025

Related U.S. Application Data
(60) Provisional application No. 63/671,732, filed on Jul. 15, 2024.

(51) Int. Cl. A61B 8/06; A61B 8/00; A61B 8/08

(52) U.S. Cl. CPC A61B 8/06; A61B 8/085; A61B 8/0891; A61B 8/4236; A61B 8/4461; A61B 8/4494; A61B 8/488; A61B 8/5223; A61B 8/54

(57) ABSTRACT

An ultrasonic sensing system including a substrate, a first linear array of ultrasonic transducers coupled to the substrate, a second linear array of ultrasonic transducers coupled to the substrate, and hardware componentry for controlling transmission of ultrasonic signals at the first linear array of ultrasonic transducers and the second linear array of ultrasonic transducers and for controlling receipt of reflected ultrasonic signals at the first linear array of ultrasonic transducers and the second linear array of ultrasonic transducers. The first linear array of ultrasonic transducers and the second linear array of ultrasonic transducers are fixedly positioned in parallel in a longitudinal direction at a fixed separation distance. The first linear array of ultrasonic transducers and the second linear array of ultrasonic transducers are positioned such that the ultrasonic signals a fixed angle and intersect at a fixed transmission distance.

### RELATED APPLICATION

This application claims priority to and the benefit of co-pending U.S. Provisional Patent Application 63/671,732, filed on Jul. 15, 2024, entitled “PORTABLE PMUT SYSTEM FOR BLOOD FLOW VELOCITY AND BLOOD VOLUME FLOW MEASUREMENT,” by Ramalli et al., having Attorney Docket No. IVS-1134-PR, and assigned to the assignee of the present application, which is incorporated herein by reference in its entirety.

### BACKGROUND

Monitoring of the cardiovascular system is often useful in the clinical evaluation of various health conditions, such as grading stenoses, controlling the quality of the vascular access for hemodialysis patients, evaluating the circulation within the foot and the leg of diabetic patients and post-operative monitoring. For these and other health conditions, continuous monitoring of cardiovascular parameters, such as blood flow volume (BFV), provides significant improvement in the clinical condition over sporadic or periodic monitoring. For instance, conventional technology in the measurement of BFV requires the use of expensive and bulky specialty instrumentation (e.g., a clinical ultrasound cart or a magnetic resonance imager) used by trained personnel in a medical facility for single or few spot-check measurements, at a significant cost for the healthcare system and inefficient time management for both patients and clinic operators. Moreover, due to technical constraints, quantitative measurements are also prone to inter-operator and interfacility value and reproducibility variations, requiring significant time of an expert sonographer to properly minimize these issues.

### BRIEF DESCRIPTION OF THE DRAWINGS

The accompanying drawings, which are incorporated in and form a part of the Description of Embodiments, illustrate various non-limiting and non-exhaustive embodiments of the subject matter and, together with the Description of Embodiments, serve to explain principles of the subject matter discussed below. Unless specifically noted, the drawings referred to in this Brief Description of Drawings should be understood as not being drawn to scale and like reference numerals refer to like parts throughout the various figures unless otherwise specified.

FIG. 1 illustrates a block diagram of an example system for cardiovascular monitoring, according to some embodiments.

FIG. 2 illustrates a block diagram of an example ultrasonic sensing module of a system for cardiovascular monitoring, according to an embodiment.

FIGS. 3A and 3B illustrate different views of an ultrasonic sensing module including two one-dimensional arrays of ultrasonic transducers, according to an embodiment.

FIGS. 4A, 4B, and 4C illustrate different views of a graphical representation of an ultrasonic sensing system including two linear arrays of ultrasonic transducers for performing cardiovascular monitoring positioned transverse to the target blood vessel, according to embodiments.

FIG. 5A illustrates a graphical representation of an example blood flow velocity determination using an ultrasonic sensing system including two linear arrays of ultrasonic transducers, according to embodiments.

FIG. 5B illustrates a graphical representation of automatic alignment of an array of ultrasonic transducers, according to embodiments.

FIGS. 6A and 6B illustrate different graphs of a cardiac cycle, according to embodiments.

FIGS. 7A and 7B illustrate different views of a graphical representation of an ultrasonic sensing system including two linear arrays of ultrasonic transducers for performing cardiovascular monitoring positioned longitudinal to the target blood vessel, according to embodiments.

FIGS. 8A and 8B illustrate cross-sectional views of ultrasonic sensing system including more than two linear arrays of ultrasonic transducers, according to some embodiments.

FIG. 9 illustrates a graphical representation of the placement of an ultrasonic sensing module, connection cable, and processing unit on a human body, according to some embodiments.

FIGS. 10A and 10B illustrate graphical representations of probe housings including an ultrasonic sensor module, according to some embodiments.

### DESCRIPTION OF EMBODIMENTS

The following Description of Embodiments is merely provided by way of example and not of limitation. Furthermore, there is no intention to be bound by any expressed or implied theory presented in the preceding background or in the following Description of Embodiments.

Reference will now be made in detail to various embodiments of the subject matter, examples of which are illustrated in the accompanying drawings. While various embodiments are discussed herein, it will be understood that they are not intended to limit to these embodiments. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding of embodiments of the present subject matter. However, embodiments may be practiced without these specific details. In other instances, well-known methods, procedures, components, and circuits have not been described in detail as not to unnecessarily obscure aspects of the described embodiments.

### Notation and Nomenclature

Some portions of the detailed descriptions which follow are presented in terms of procedures, logic blocks, processing and other symbolic representations of operations on data within an electrical device. These descriptions and representations are the means used by those skilled in the data processing arts to most effectively convey the substance of their work to others skilled in the art. In the present application, a procedure, logic block, process, or the like, is conceived to be one or more self-consistent procedures or instructions leading to a desired result. The procedures are those requiring physical manipulations of physical quantities. Usually, although not necessarily, these quantities take the form of acoustic (e.g., ultrasonic) signals capable of being transmitted and received by an electronic device and/or electrical or magnetic signals capable of being stored, transferred, combined, compared, and otherwise manipulated in an electrical device.

It should be borne in mind, however, that all of these and similar terms are to be associated with the appropriate physical quantities and are merely convenient labels applied to these quantities. Unless specifically stated otherwise as apparent from the following discussions, it is appreciated that throughout the description of embodiments, discussions utilizing terms such as “controlling,” “performing,” “determining,” “detecting,” “sensing,” “transmitting,” “processing,” “providing,” “receiving,” “analyzing,” “confirming,” “using,” “completing,” “instructing,” “comparing,” “correlating,” “executing,” or the like, refer to the actions and processes of an electronic device such as an ultrasonic sensing system.

Embodiments described herein may be discussed in the general context of processor-executable instructions residing on some form of non-transitory processor-readable medium, such as program modules, executed by one or more computers or other devices. Generally, program modules include routines, programs, objects, components, data structures, etc., that perform particular tasks or implement particular abstract data types. The functionality of the program modules may be combined or distributed as desired in various embodiments.

In the figures, a single block may be described as performing a function or functions; however, in actual practice, the function or functions performed by that block may be performed in a single component or across multiple components, and/or may be performed using hardware, using software, or using a combination of hardware and software. To clearly illustrate this interchangeability of hardware and software, various illustrative components, blocks, modules, logic, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example ultrasonic sensing system and/or mobile electronic device described herein may include components other than those shown, including well-known components.

Various techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium comprising instructions that, when executed, perform one or more of the methods described herein. The non-transitory processor-readable data storage medium may form part of a computer program product, which may include packaging materials.

The non-transitory processor-readable storage medium may comprise random access memory (RAM) such as synchronous dynamic random access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, and/or executed by a computer or other processor.

Various embodiments described herein may be executed by one or more processors, such as one or more motion processing units (MPUs), sensor processing units (SPUs), host processor(s) or core(s) thereof, digital signal processors (DSPs), general purpose microprocessors, application specific integrated circuits (ASICs), application specific instruction set processors (ASIPs), field programmable gate arrays (FPGAs), a programmable logic controller (PLC), a complex programmable logic device (CPLD), a discrete gate or transistor logic, discrete hardware components, or any combination thereof designed to perform the functions described herein, or other equivalent integrated or discrete logic circuitry. The term “processor,” as used herein may refer to any of the foregoing structures or any other structure suitable for implementation of the techniques described herein. As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Moreover, processors can exploit nano-scale architectures such as, but not limited to, molecular and quantum-dot based transistors, switches and gates, in order to optimize space usage or enhance performance of user equipment. A processor may also be implemented as a combination of computing processing units.

In addition, in some aspects, the functionality described herein may be provided within dedicated software modules or hardware modules configured as described herein. Also, the techniques could be fully implemented in one or more circuits or logic elements. A general purpose processor may be a microprocessor, but in the alternative, the processor may be any conventional processor, controller, microcontroller, or state machine. A processor may also be implemented as a combination of computing devices, e.g., a combination of an SPU/MPU and a microprocessor, a plurality of microprocessors, one or more microprocessors in conjunction with an SPU core, MPU core, or any other such configuration.

### Overview of Discussion

Medical ultrasound technology is currently employed by medical professionals for imaging of the vascular system. Based on the images, medical professionals, such as ultrasound technicians, can deduce various forms of information regarding vascular health, such as vascular wall motion, blood flow, or elastic properties of the soft tissues (e.g., elastography). Typical ultrasound systems currently in use are for clinical usage and are meant to be operated by specially educated medical experts. Conventional medical ultrasonic systems typically include ultrasound probes with various shapes and form factors for different body parts and often output images that are then analyzed further. In a clinical setting, the ultrasound system is used by a physician or clinician to align the probes to the physiological sites of interest and diagnose based on the static ultrasound imaging, Doppler imaging, and elastography. Due to the complexity of the biological system and usage of the ultrasound systems, extensive ultrasound imaging and medical training are needed for conventional ultrasound examination and diagnosis.

Technology development over the last decades has resulted in miniaturized ultrasonic transducers as well as ever-increasing data processing power and storage. An example of the currently available miniaturized ultrasonic transducers is the application of ultrasonic fingerprint sensors in mobile devices. Embodiments described herein provide an ultrasonic sensor system for cardiovascular system monitoring. The described system provides a user-friendly system that does not necessarily require operation by a trained medical professional, but due to system optimization and signal processing, allows for home usage outside of a medical establishment. For example, the described system can be used by people at home (without medical training), by in-home care personnel, or even by automated home robots or similar autonomous devices. The described system can measure and output various parameters of the blood vessels, e.g., pulse wave velocity (PWV), blood flow, blood flow volume (BFV), etc. For example, accurate, reliable, and noninvasive measurement of BFV in blood vessels provides clinically relevant information in a variety of situations such as grading stenoses, controlling the quality of the vascular access for hemodialysis patients, evaluating the circulation within the foot and the leg of diabetic patients and post- operative monitoring.

Embodiments described herein provide an ultrasonic sensing system including a substrate, linear arrays of ultrasonic transducers, and hardware componentry for controlling transmission of the ultrasonic signals and receipt of reflected ultrasonic signals at the arrays of ultrasonic transducers. The ultrasonic sensing system is configured to be positioned on a body adjacent to a blood vessel to perform cardiovascular monitoring. The linear arrays of ultrasonic transducers are coupled to the substrate at a known angle relative to each other such that the ultrasonic signals intersect at a known point or line within body tissue, thereby enabling continuous cardiovascular monitoring using triangulation Doppler analysis. In some embodiments, the substrate and linear arrays of ultrasonic transducers are comprised within a wearable patch that is configured to be attached to a body proximate a blood vessel. In other embodiments, the substrate and linear arrays of ultrasonic transducers are comprised within a probe housing that is configured to be manually positioned on a body proximate a blood vessel.

By enabling continuous monitoring of cardiovascular parameters, such as BFV, the described embodiments improve the clinical decisions and reduce both the operator time and the operator dependency. In accordance with some embodiments, the described system processes the data received from the arrays using triangulation Doppler methods. The use of ultrasonic transducers, such as a piezoelectric micromachined ultrasonic transducers (pMUTs), allows for usage within a wearable patch (e.g., comprised of a flexible material) to replace conventional medical ultrasound probes, thereby enabling a wearable monitor that can insonicate the blood vessel at the correct angles without patient discomfort for prolonged time.

By accurately measuring the BFV within a small form factor system that can be worn by a patient, the described embodiments also provide for measurement of the local pulse wave velocity (PWV) on the blood vessel of the patient by exploiting the flow and area (QA) method. PWV is a critical parameter to understand the vascular age of a person, a parameter whose importance is growing both within the medical community and within the consumer wellness market. The PWV can also correlate with blood pressure to realize a cuffless wearable blood pressure monitor. The improved accuracy given by system of the described embodiments and the unique geometries that can be realized with pMUTs on substrates within wearable patches also enables the design of specialized instruments to assess the blood flow that can have superior performance compared to standard ultrasound systems allowing more rapid and precise assessment in several clinical exams, for example, to stage vascular problems in the diabetic foot.

Embodiments described ultrasonic sensing system include a substrate, a first linear array of ultrasonic transducers coupled to the substrate for transmitting ultrasonic signals in a first direction, a second linear array of ultrasonic transducers coupled to the substrate for transmitting ultrasonic signals in a second direction, and hardware componentry for controlling transmission of the ultrasonic signals at the first linear array of ultrasonic transducers and the second linear array of ultrasonic transducers and for controlling receipt of reflected ultrasonic signals at the first linear array of ultrasonic transducers and the second linear array of ultrasonic transducers. The first linear array of ultrasonic transducers and the second linear array of ultrasonic transducers are fixedly positioned in parallel in a longitudinal direction at a fixed separation distance, and the first linear array of ultrasonic transducers and the second linear array of ultrasonic transducers are positioned such that the ultrasonic signals transmitted in the first direction intersect with the ultrasonic signals transmitted in the second direction at a fixed angle and intersect at a fixed transmission distance. The substrate allows for the transmission of ultrasonic signals within tissue in contact with the ultrasonic sensing system and holds the arrays at a fixed inter-probe distance with a fixed relative angle. Each array of ultrasonic transducers is used to generate an image of the target blood vessel on different cross-sectional planes, with the planes interesting on a line at a known depth and position from the sensor surface, for example, within a target blood vessel.

In some embodiments, the ultrasonic sensing system further includes a wearable patch configured to be attached to a body proximate a blood vessel, wherein the substrate, the first linear array of ultrasonic transducers and the second linear array of ultrasonic transducers are coupled to the wearable patch and positioned such that the first linear array of ultrasonic transducers and the second linear array of ultrasonic transducers can project ultrasonic signals into the body.

In some embodiments, the ultrasonic sensing system further includes a probe housing configured to be manually positioned on a body proximate a blood vessel, wherein the substrate, the first linear array of ultrasonic transducers and the second linear array of ultrasonic transducers are coupled to the probe housing and positioned such that the first linear array of ultrasonic transducers and the second linear array of ultrasonic transducers can project ultrasonic signals into the body. In some embodiments, the ultrasonic sensing system further includes a mechanical control mechanism disposed within the probe housing and movably coupled to the first linear array of ultrasonic transducers and the second linear array of ultrasonic transducers, the mechanical control mechanism configured to control the fixed angle of the ultrasonic signals transmitted in the first direction and the ultrasonic signals transmitted in the second direction.

In some embodiments, the ultrasonic sensing system further includes a third linear array of ultrasonic transducers coupled to the substrate, the third linear array of ultrasonic transducers for transmitting ultrasonic signals that intersect with the ultrasonic signals transmitted from the second linear array of ultrasonic signals transmitted in the second direction. The third linear array of ultrasonic transducers is fixedly positioned in parallel in the longitudinal direction to the first linear array of ultrasonic transducers and the second linear array of ultrasonic transducers and at a second fixed separation distance from the second linear of ultrasonic transducers, and wherein the third linear array of ultrasonic transducers and the second linear array of ultrasonic transducers are positioned such that the ultrasonic signals transmitted from the third linear array intersect with the ultrasonic signals transmitted from the second linear array in the second direction at a second fixed angle and intersect at a second fixed transmission distance. In some embodiments, the third linear array of ultrasonic transducers is positioned for transmitting ultrasonic signals in the first direction, wherein the second fixed angle is equal to the fixed angle, and wherein the third linear array of ultrasonic transducers and the second linear array of ultrasonic transducers are positioned such that the ultrasonic signals transmitted from the third linear array in the first direction intersect with the ultrasonic signals transmitted from the second linear array at the second fixed transmission distance.

In some embodiments, the ultrasonic sensing system further includes a fourth linear array of ultrasonic transducers coupled to the substrate, the fourth linear array of ultrasonic transducers for transmitting ultrasonic signals that intersect with the ultrasonic signals transmitted from the first linear array of ultrasonic signals transmitted in the first direction. The fourth linear array of ultrasonic transducers is fixedly positioned in parallel in the longitudinal direction to the first linear array of ultrasonic transducers and the second linear array of ultrasonic transducers and at a third fixed separation distance from the first linear of ultrasonic transducers, and wherein the fourth linear array of ultrasonic transducers and the first linear array of ultrasonic transducers are positioned such that the ultrasonic signals transmitted from the fourth linear array intersect with the ultrasonic signals transmitted from the first linear array in the first direction at a third fixed angle and intersect at a third fixed transmission distance. In some embodiments, the fourth linear array of ultrasonic transducers is positioned for transmitting ultrasonic signals in the second direction, wherein the third fixed angle is equal to the fixed angle, and wherein the fourth linear array of ultrasonic transducers and the first linear array of ultrasonic transducers are positioned such that the ultrasonic signals transmitted from the fourth linear array in the second direction intersect with the ultrasonic signals transmitted from the first linear array at the third fixed transmission distance.

In some embodiments, the ultrasonic sensing system further includes a processing unit for processing the reflected ultrasonic signals received at the first linear array of ultrasonic transducers and the second linear array of ultrasonic transducers for performing cardiovascular monitoring.

In some embodiments, the first linear array of ultrasonic transducers and second linear array of ultrasonic transducers are configured to transmit ultrasonic signals into a blood vessel such that the ultrasonic signals intersect at an intersection point at the fixed transmission distance within the blood vessel. The processing unit is configured to determine mean phase changes of consecutive instances of the reflected ultrasonic signals received at the first linear array of ultrasonic signals and the second linear array of ultrasonic signals, is configured to determine blood flow velocity at the intersection point based at least in part on the mean phase changes, and is configured to determine blood flow direction and Doppler angle at the intersection point by using triangulation to extract blood velocity components from the mean phase changes of consecutive instances of the reflected ultrasonic signals.

In some embodiments, the processing unit is configured to generate a Color Flow Doppler (CFD) image of the blood flow velocity within the blood vessel using the Doppler angle derived at least in part on using triangulation. In some embodiments, the processing unit is configured to calculate blood flow volume from the blood flow velocity within a lumen area of the blood vessel. In some embodiments, the processing unit is configured to determine the lumen area based at least in part on using the reflected ultrasonic signals received at the first linear array of ultrasonic transducers and the second linear array of ultrasonic transducers to determine wall position of the blood vessel over progressive instances of the reflected ultrasonic signals. In some embodiments, the processing unit is configured to determine vessel wall position using at least an imaging mode to extract the blood flow velocity over an outer region of the lumen area using interpolation and over progressive instances of the reflected ultrasonic signals. In some embodiments, the processing unit is configured to determine a pulse wave velocity within the blood vessel based at least in part on the blood flow velocity and the lumen area.

In some embodiments, the processing unit is configured to determine a lumen area based at least in part on using the reflected ultrasonic signals received at the first linear array of ultrasonic transducers and the second linear array of ultrasonic transducers to determine wall position of a blood vessel.

In some embodiments, the processing unit is configured to perform an imaging mode to obtain morphological images of a blood vessel based at least in part on the reflected ultrasonic signals received at the first linear array of ultrasonic transducers and the second linear array of ultrasonic transducers.

In some embodiments, the processing unit is configured to perform alignment of the ultrasonic signals transmitted by the first linear array of ultrasonic transducers and second linear array of ultrasonic transducers to identify a line aligned with a center of a blood vessel. In some embodiments, the processing unit is configured to estimate a blood flow velocity profile at the line within the blood vessel.

In some embodiments, the processing unit is configured to generate a Color Flow Doppler (CFD) line of the blood flow velocity within the blood vessel using the Doppler angle derived at least in part on using triangulation. In some embodiments, the processing unit is configured to calculate blood flow volume from the estimate of the blood flow velocity profile at the line within the blood vessel. In some embodiments, the processing unit is configured to determine a lumen area based at least in part on using the reflected ultrasonic signals received at the first linear array of ultrasonic transducers and the second linear array of ultrasonic transducers to determine wall position of the blood vessel at the line over progressive instances of the reflected ultrasonic signals and extrapolating to determine the lumen area. In some embodiments, the processing unit is configured to determine vessel wall position using at least an imaging mode to extract blood flow velocity over an outer region of the lumen area using interpolation and over progressive instances of the reflected ultrasonic signals.

### EXAMPLE ULTRASONIC SENSOR SYSTEM FOR CARDIOVASCULAR MONITORING

Embodiments described herein provide an ultrasonic sensing system for acquiring cross-section planes of a blood vessel, crossing each other within the blood vessel, allowing for the extraction of the blood flow volume (BFV) and the cross-sectional blood vessel diameter variation at the same time using Doppler triangulation. In some embodiments, the ultrasonic sensing system is integrated within a wearable device (e.g., a flexible patch) that allows for small device dimensions that adapt geometry to a desired position on a human body for transmitting ultrasonic signals towards a target blood vessel within the body. The use of ultrasonic transducer arrays on a flexible material for coupling to a human body makes it possible to realize geometries for Doppler triangulation for positioning the ultrasonic sensing system in contact with the skin on the acquisition location (e.g., the neck to monitor the carotid artery).

Embodiments described herein provide ultrasonic sensing systems for cardiovascular monitoring, including a substrate, a first linear array of ultrasonic transducers coupled to the substrate, a second linear array of ultrasonic transducers coupled to the substrate, and hardware componentry for controlling transmission of a ultrasonic signals at the first linear array of ultrasonic transducers and the second linear array of ultrasonic transducers and for controlling receipt of reflected ultrasonic signals at the first linear array of ultrasonic transducers and the second linear array of ultrasonic transducers. The first linear array of ultrasonic transducers is for transmitting ultrasonic signals in a first direction, and the second linear array of ultrasonic transducers is for transmitting ultrasonic signals in a second direction. The first linear array of ultrasonic transducers and the second linear array of ultrasonic transducers are fixedly positioned in parallel in a longitudinal direction at a fixed separation distance, wherein the first linear array of ultrasonic transducers and the second linear array of ultrasonic transducers are positioned such that the ultrasonic signals transmitted in the first direction intersect with the ultrasonic signals transmitted in the second direction at a fixed angle and intersect at a fixed transmission distance.

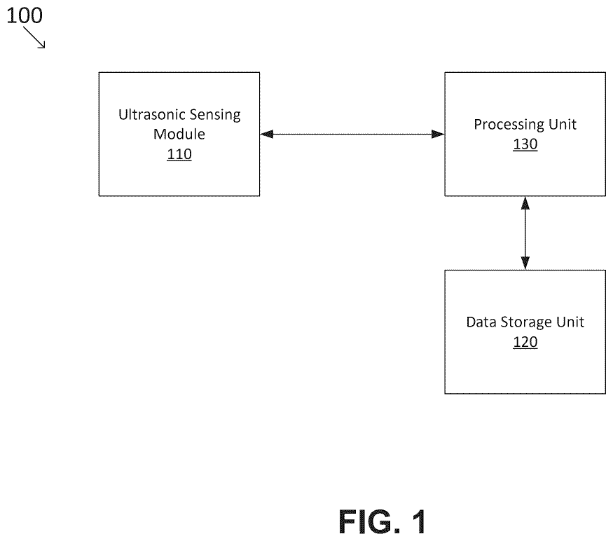

FIG. 1 illustrates a block diagram of an example ultrasonic sensing system 100 for cardiovascular monitoring, according to some embodiments. System 100 is configured to perform cardiovascular monitoring based at least in part on signals (e.g., acoustic signals) received from an ultrasonic sensor. Ultrasonic sensing system 100 includes ultrasonic sensing module 110, data storage unit 120, and processing unit 130. In some embodiments, ultrasonic sensing module 110 and processing unit 130 are communicatively coupled via a connection cable or connection cable assembly. Ultrasonic sensor module 110 is communicatively coupled to processing unit 130 and enables signal communication and power transmission between ultrasonic sensor module 110 and processing unit 130. While data storage unit 120 is illustrated as being external to processing unit 130, it should be appreciated that in some embodiments, data storage unit 120 may be integrated within processing unit 130. In other embodiments, data storage unit 120 may be distributed such that a portion of data storage unit 120 is integrated within processing unit 130 and a portion of data storage unit 120 is external to processing unit 130.

Ultrasonic sensing module 110 is for placement on a human body proximate a blood vessel for performing cardiovascular monitoring. In some embodiments, the cardiovascular monitoring includes at least one of pulse wave velocity, pulse transit time, arterial diameter, arterial wall motion, arterial wall stiffness, heart rate, blood flow volume, and blood pressure. In accordance with the described embodiments, ultrasonic sensing module 110 includes at least two arrays of ultrasonic transducers, where the arrays of ultrasonic transducers include a plurality of ultrasonic transducers. In some embodiments, the arrays of ultrasonic transducers are one-dimensional arrays.

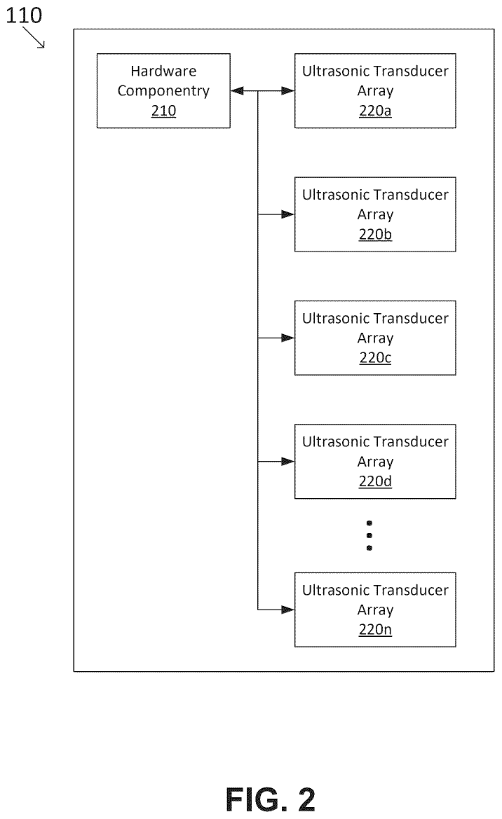

System 100 includes hardware componentry for controlling transmission of ultrasonic signals at ultrasonic sensing module 110 and for controlling receipt of reflected ultrasonic signals at ultrasonic sensing module 110. In some embodiments (e.g., as illustrated in FIG. 2), the hardware componentry is comprised within ultrasonic sensing module 110. In other embodiments, the hardware componentry is comprised within processing unit 130. System 100 may also include other components that are not illustrated, so as to not obfuscate the described embodiments, but are well understood to those of skill in the art, such as pre-amplifiers, filters, analog-to-digital converters, a digital processing module for performing on-board signal processing of the reflected ultrasonic signals, and a power control system including an energy storage device (e.g., a battery, a super capacitor, etc.) for providing power to the hardware componentry, the digital processing module, and ultrasonic sensor module 110. Processing unit 130 is configured for processing the reflected ultrasonic signals received at arrays of ultrasonic sensing module 110.

FIG. 2 illustrates a block diagram of an example ultrasonic sensing module 110 of a system for cardiovascular monitoring, according to an embodiment. Ultrasonic sensing module 110 is for placement on a human body proximate a blood vessel for performing cardiovascular monitoring.

With reference to FIG. 2, ultrasonic sensing module 110 includes hardware componentry 210 and a plurality of ultrasonic transducer arrays 220a-220n (individually referred to herein as ultrasonic transducer arrays 220). It should be appreciated that ultrasonic sensing module 110 can include two or more ultrasonic transducer arrays under the control of hardware componentry 210. At least two ultrasonic transducer arrays 220 are positioned such that ultrasonic signals transmitted from the two ultrasonic transducer arrays 220 intersect at a fixed angle relative to each other and intersect at a fixed transmission distance.

Hardware componentry 210 is for controlling transmission of the ultrasonic signals at the first linear array of ultrasonic transducers and the second linear array of ultrasonic transducers and for controlling receipt of reflected ultrasonic signals at the first linear array of ultrasonic transducers and the second linear array of ultrasonic transducers. It should be appreciated that hardware componentry 210 can include a pre-amplification device, secondary sensors such as a motion sensor, a force sensor, or a temperature sensor, and/or an alignment light emitting diode (LED) for assisting in visual alignment of ultrasonic sensing module 110 with a blood vessel on a human body.

Hardware control componentry 210 is configured to control operation of ultrasonic sensing module 110. For instance, hardware control componentry 210 is configured to communicate sensor control signals to ultrasonic transducer arrays 220a-220n to control transmission of ultrasonic signals at ultrasonic transducer arrays 220a-220n and to control receipt of reflected ultrasonic signals at ultrasonic transducer arrays 220a-220n. It should be appreciated that hardware control componentry 210 can perform other control operations, such as signal amplification, analog to digital conversion, and other functionality. Received signals are received at hardware control componentry 210 for communication and analysis, e.g., by processing unit 130 or by a digital processing module. Hardware control componentry 210 includes voltage transmitters to drive the ultrasonic sensing module, controlling signal transmission strength, frequency, and activation as sensor control signals.

Hardware control componentry 210 is configured to activate ultrasonic transducers of at least one ultrasonic sensor (e.g., an array of ultrasonic transducers) to perform ultrasonic signal transmission and receipt of reflected ultrasonic signals. In some embodiments, hardware control componentry 210 transmits sensor control signals for individually activating and operating ultrasonic transducers of an ultrasonic transducer array. In other embodiments, hardware control componentry 210 transmits sensor control signals for collectively operating a subset of ultrasonic transducers of an ultrasonic transducer array to perform beamforming and/or beam steering of an ultrasonic beam. For instance, sensor control signals may delay activation of some ultrasonic transducers of an ultrasonic transducer array relative to other ultrasonic transducers, to focus a transmit beam to a particular location on or within the human body.

Ultrasonic transducer arrays 220 are operable to emit and detect ultrasonic waves (also referred to as ultrasonic signals or ultrasound signals). One or more ultrasonic transducers (e.g., Piezoelectric Micromachined Ultrasonic Transducers (PMUTs)), which may be comprised within ultrasonic transducer array 220 may be used to transmit and receive the ultrasonic waves, where the ultrasonic transducers perform both the transmission and receipt of the ultrasonic waves. The emitted ultrasonic waves are reflected from any objects in contact with (or in front of) ultrasonic transducer array 220, and can project into the object at various depths, and these reflected ultrasonic waves, or echoes, are then detected and received at ultrasonic transducer array 220 as received ultrasonic signals (e.g., reflected ultrasonic signals). Where the object is a human body (e.g., at an arm or a wrist), the waves are projected into the tissue of the human body and reflect at different tissue depths due to acoustic impedance mismatches.

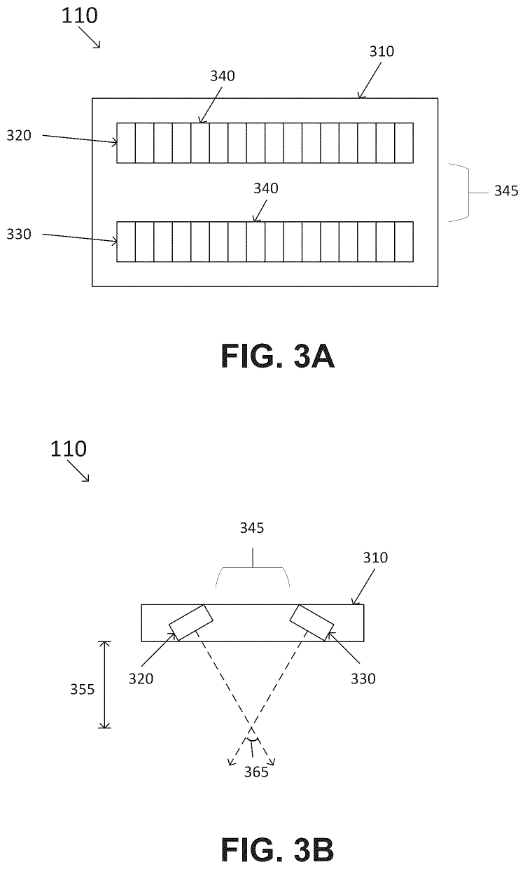

In some embodiments, ultrasonic transducer array 220 is a one-dimensional array of ultrasonic transducers. In some embodiments, ultrasonic transducer array 220 is a two-dimensional array of ultrasonic transducers. FIGS. 3A and 3B illustrate different views of an ultrasonic sensing module 110 including two one-dimensional arrays of ultrasonic transducers, according to an embodiment.

FIG. 3A illustrates a top view of ultrasonic sensing module 110 including two one-dimensional linear arrays 320 and 330 of ultrasonic transducers 340. It should be appreciated that ultrasonic transducers 340 can be of any shape or size, e.g., square, rectangular, circular, etc. One-dimensional linear arrays 320 and 330 are coupled to substrate 310. Substrate 310 is configured to fixedly hold one-dimensional linear arrays 320 and 330 in parallel in a longitudinal direction at a fixed separation distance 345 from each other. Substrate 310 (also referred to herein as “coupling substrate” or “acoustic coupling substrate”) allows for the transmission of ultrasonic signals between ultrasonic transducers of the one-dimensional linear arrays 320 and 330 and the tissue in contact with ultrasonic sensing module 110.

FIG. 3B illustrates an end view of ultrasonic sensing module 110 including two one-dimensional linear arrays 320 and 330 of ultrasonic transducers 340. As illustrated in FIG. 3B, one-dimensional linear arrays 320 and 330 are angled relative to each other such that ultrasonic signals transmitted from one-dimensional linear arrays 320 and 330 are transmitted at a fixed angle 365 relative to each other and intersect at a fixed transmission distance 355. It should be appreciated that during operation one- dimensional linear arrays 320 and 330 are fixedly positioned such that fixed angle 365 and transmission distance 355 are known. In some embodiments, such as where ultrasonic sensing module 110 is implemented within a handheld probe, the handheld probe may include a mechanical control mechanism that allows for moving of the one-dimensional linear arrays 320 and 330 for controlling fixed angle 365.

In various embodiments, one-dimensional linear arrays 320 and 330 may be used for forming and steering an ultrasonic beam. The beam forming can be used to focus the ultrasonic waves at the correct depth, and the beam steering may be used to control the lateral position of the beam to locate the blood vessel. For example, when the sensor is placed on the skin, the sensor may not be exactly above the blood vessel. The beam steering and beamforming may be used to locate the vessel in a first step through a scanning action, and once the vessel is located, in a second step, perform the blood vessel and blood flow measurements. The beam forming and beam steering can be accomplished by applying small phase or time delays to the individual transducers. The ultrasonic transducers may be controlled individually, or the ultrasonic transducers may be grouped together in subsets of transducers. These subsets of ultrasonic transducers may be independently and/or collectively controlled. This may be done for generating the ultrasonic beam (transmit beamforming), but it may also help with the signal analysis of the detected reflected waves (receive beamforming). Location of the blood vessel may also be based on Doppler measurements or by looking for signal with the right heartbeat signal or frequency components. Furthermore, optimizing for a maximum change in amplitude can be used to determine the center middle of the blood vessel.

One-dimensional linear arrays 320 and 330 operable to emit and detect ultrasonic waves (also referred to as ultrasonic signals or ultrasound signals). One or more ultrasonic transducers (e.g., PMUTs), which may be comprised within one- dimensional linear arrays 320 and 330, may be used to transmit and receive the ultrasonic waves, where the ultrasonic transducers are capable of performing both the transmission and receipt of the ultrasonic waves. The emitted ultrasonic waves are reflected from any objects in contact with (or in front of) one-dimensional linear arrays 320 and 330, and can project into the object at various depths, and these reflected ultrasonic waves, or echoes, are then detected and received at one-dimensional linear arrays 320 and 330 as received signals. Where the object is a human body (e.g., at an arm or a wrist), the waves are projected into the tissue of the human body, and reflect at different tissue depths due to acoustic impedance mismatches.

The system for cardiovascular monitoring described herein is capable of being worn on the human body as a wearable system that allows for continuous capture of ultrasound measurements for prolonged times. For example, the wearable system for cardiovascular monitoring described herein can be worn in the same fashion as cardiac Holter monitors, ambulatory blood pressure monitoring systems or continuous pulse oximeters, also enabling spot-check measurements as many of these other monitoring systems do. The described embodiments can require little user interaction during use, and are capable of continuous monitoring, periodic monitoring, and also enabling beat-to-beat information retrieval during cardiovascular monitoring.

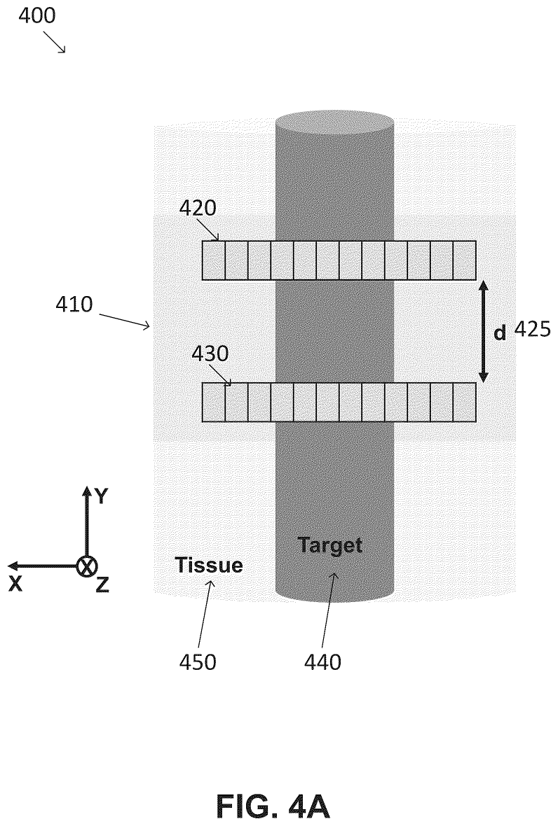

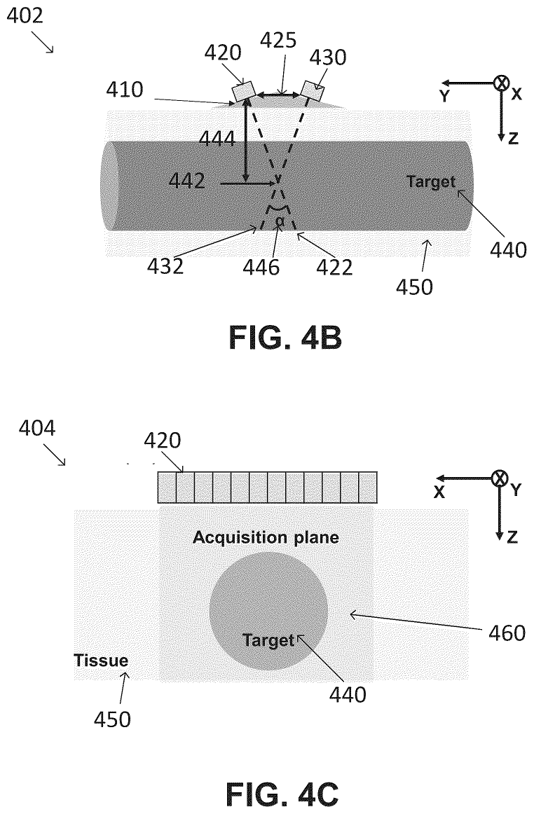

FIGS. 4A, 4B, and 4C illustrate different views of a graphical representation of an ultrasonic sensing system 410 including two linear arrays 420 and 430 of ultrasonic transducers for performing cardiovascular monitoring positioned transverse to the target blood vessel 440 within tissue 450, according to embodiments. FIG. 4A illustrates a top view 400 of ultrasonic sensing system 410. Linear arrays 420 and 430 are in parallel in a longitudinal direction at a fixed separation distance (d) 425 from each other. Ultrasonic sensing system 410 allows for the transmission of ultrasonic signals between ultrasonic transducers of the linear arrays 420 and 430 and tissue 450 in contact with ultrasonic sensing system 410 for sensing target blood vessel 440. It should be appreciated that ultrasonic sensing system 410 can be placed in any position relative to target blood vessel 440, such as transversally or longitudinally depending on the information that needs to be extracted and with some angular tolerance.

FIG. 4B illustrates a side view 402 of ultrasonic sensing system 410. Ultrasonic signals 422 are transmitted from linear array 420 and ultrasonic signals 432 are transmitted from linear array 430 when ultrasonic sensing system 410 is in contact with tissue 450. Linear array 420 and linear array 430 are separated at fixed separation distance 425, and have a fixed relative angle (a) 446, such that ultrasonic signals 422 and 432 intersect at intersection line 442, which is at fixed transmission distance 444 from ultrasonic sensing system 410. Linear array 420 and linear array 430 each image target blood vessel 440 on a different cross-sectional plane and the two planes intersect at intersection line 442 that is at fixed transmission distance 444 from the sensor surface. Fixed transmission distance (zᵢ) 444 is determined as shown in Equation 1:

$${Z}_{i} = \frac{d}{2 * tan ( \frac{α}{2} )} \qquad ( 1 )$$

Ultrasonic sensing system 410 is configured to have the center of the target blood vessel 440 on or in close proximity to intersection line 442. For example, in the case of the carotid artery, a possible configuration is zᵢ=2 cm, d=1.45 cm and α=40°. While this can also be realized with traditional medical probes using a suitable holder, the form factor can create significant discomfort for the patient when in place, e.g., on the neck of the subject, and cannot be easily held in position by a sonographer. Using ultrasonic sensors to implement ultrasonic sensing system 410 it is possible to realize the ultrasonic sensing system 410 in a patch form factor that can be easily worn by the patient for long term monitoring and that can simplify its use also by the sonographer. The geometry becomes more and more disadvantageous or impossible to achieve with conventional medical probes the shallower is the target blood vessel.

FIG. 4C illustrates an end view 404 of ultrasonic sensing system 410. As illustrated in view 404, acquisition plane 460 that is imaged at linear array 420 images a cross-section of target blood vessel 440. Linear array 430 also images a cross-section of target blood vessel 440.

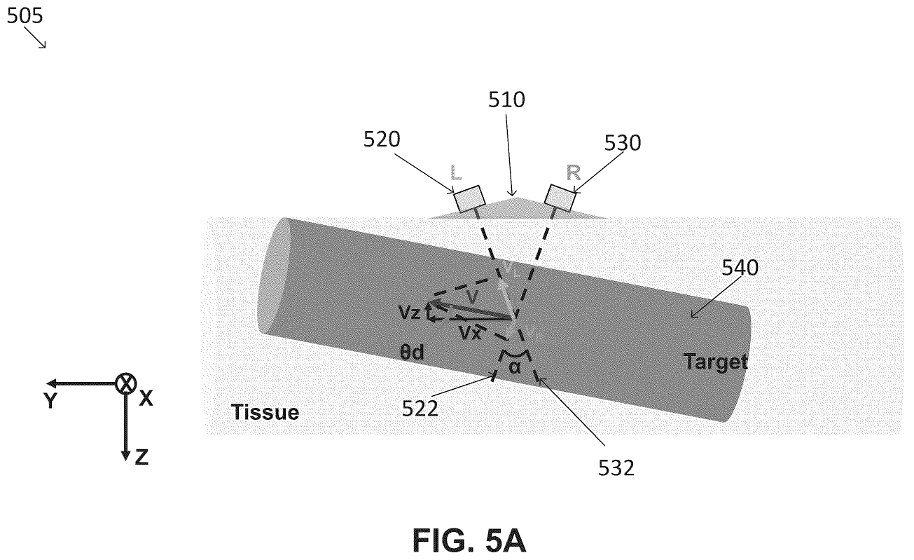

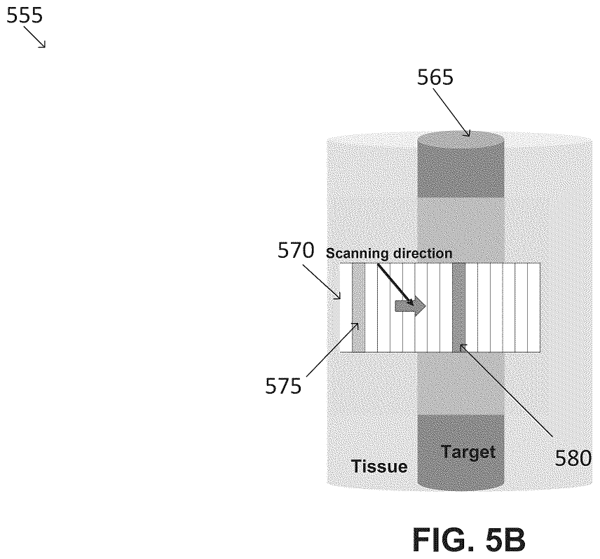

FIG. 5A illustrates a graphical representation of a side view of an example blood flow velocity determination 505 using an ultrasonic sensing system 510 including two linear arrays 520 and 530 of ultrasonic transducers oriented transversally with respect to the blood vessel, according to embodiments. The described embodiments provide for obtaining the blood flow velocity (BFV) profile on the blood vessel lumen section using ultrasound Doppler techniques. The blood vessel is imaged by each array using high frame rate plane wave imaging enabling a sufficient temporal resolution to discern the blood flow velocity along the different phases of the cardiac cycle. The in-phase and quadrature data (IQ data) are processed to obtain a Color Flow Doppler (CFD) image with high temporal resolution from each array by applying first a clutter filter (e.g. an Infinite Impulse Response (IIR) high-pass filter (HPF)) to attenuate the slowly moving tissue signals and then extracting the signal phase changes Δφ with the autocorrelation Kasai's method. This signal phase change can be converted into actual flow velocity if the Doppler angle is known using the following formula (Equation 2):

$$V = \frac{c {f}_{d}}{2 {f}_{0} cos {θ}_{d}} = \frac{{c Δ φ f}_{PRF}}{4 π cos {θ}_{d}} \qquad ( 2 )$$

where V is the blood velocity, c is the speed of sound in tissue, f_{d} is the Doppler frequency shift, f₀ is the transmitting frequency and θ_{d} is the angle of incidence between the ultrasound beam and the direction of flow. In conventional pulsed wave Doppler the angle is assigned manually by the operator with significant errors in the evaluation of V if θ_{d} is close to 90 degrees and therefore θ_{d} should be kept below 60° or above 120°. The use of two CFD at a known angles allows to establish, automatically, without any operator input, the Doppler angle and the absolute velocity by exploiting Doppler triangulation.

At the intersection point of the ultrasound beams of the two arrays, the echoes acquired by the left and right apertures are processed to compute the mean Doppler shifts, f_{dR} and f_{dR}, respectively, obtained along the ultrasound propagation directions. By using trigonometric triangulation (as shown in Equations 3, 4, and 5), it is possible to extract the lateral (Vₓ) and axial (V_{z}) components of the velocity vector V with respect to the apertures' position leveraging the knowledge of the angle α between the arrays. This allows for the extraction of the actual flow-to-probe angle (θ_{d}) without any assumptions on flow velocity direction and profile (given the fact that two-dimensional artery cross-sectional images are acquired), thus enabling reliable BFV estimates.

$${V}_{x} = \frac{c}{2 {f}_{0}} \frac{{f}_{dR} - {f}_{dL}}{2 sin ( \frac{α}{2} )} \qquad ( 3 )$$

$${V}_{z} = \frac{c}{2 {f}_{0}} \frac{{f}_{dR} + {f}_{dL}}{2 cos ( \frac{α}{2} )} \qquad ( 4 )$$

$${θ}_{d} = arg ( {V}_{x} , {V}_{Z} ) \qquad ( 5 )$$

Although BFV can be extracted from the CFD obtained from any of the two probes, the data coming from the probe with a bigger Doppler angle (e.g., further away from 90 degrees between flow direction and ultrasonic signal is expected to provide better estimates. In case the Doppler angle is similar for the two probes, one can use the average of the two CFD for example. The BFV is finally extracted integrating the CFD image with the most appropriate Doppler angle over the lumen area using Equation 6:

$$BFV = \int {\int }_{S} V ( x , y ) \cdot dS = \int {\int }_{S} ( \frac{{f}_{d} ( x , z ) \cdot c}{2 {f}_{0} \cdot cos {θ}_{d}} ) \cdot dS \qquad ( 6 )$$

where f_{d} is the blood Doppler frequency shift over the plane of interest, c the speed of sound, f₀ the transmitting frequency, θ_{d} the Doppler angle and S the lumen area. In some embodiments, plane wave compounding could be used instead of simple plane wave imaging at the expense of the maximum achievable frame rate and additional memory and computational costs or possibly line beamforming.

In some embodiments, linear arrays 520 and 530 are configured to transmit ultrasonic signals 522 and 532, respectively, into target blood vessel 540 such that the ultrasonic signals intersect at an intersection point at the fixed transmission distance within the target blood vessel 540. A processing unit (e.g., processing unit 130) is configured to determine mean phase changes of consecutive instances of the reflected ultrasonic signals received at linear arrays 520 and 530, is configured to determine blood flow velocity at the intersection point based at least in part on the mean phase changes, and is configured to determine blood flow direction and Doppler angle at the intersection point by using triangulation to extract blood velocity components from the mean phase changes of consecutive instances of the reflected ultrasonic signals.

In some embodiments, the processing unit is configured to generate a CFD image of the blood flow velocity within the blood vessel using the Doppler angle derived at least in part on using triangulation. In some embodiments, the processing unit is configured to calculate blood flow volume from the blood flow velocity within a lumen area of the blood vessel. In some embodiments, the processing unit is configured to determine the lumen area based at least in part on using the reflected ultrasonic signals received at linear arrays 520 and 530 to determine wall position of the blood vessel over progressive instances of the reflected ultrasonic signals. In some embodiments, the processing unit is configured to determine vessel wall position using at least an imaging mode to extract the blood flow velocity over an outer region of the lumen area using interpolation and over progressive instances of the reflected ultrasonic signals. In some embodiments, the processing unit is configured to determine a pulse wave velocity within the blood vessel based at least in part on the blood flow velocity and the lumen area.

In some embodiments, the processing unit is configured to determine a lumen area based at least in part on using the reflected ultrasonic signals received at linear arrays 520 and 530 to determine the wall position of a blood vessel. In some embodiments, the processing unit is configured to perform an imaging mode to obtain morphological images of a blood vessel based at least in part on the reflected ultrasonic signals received at linear arrays 520 and 530.

In some embodiments, the processing unit is configured to perform alignment of the ultrasonic signals transmitted by linear arrays 520 and 530 to identify a line aligned with a center of target blood vessel 540. In some embodiments, the processing unit is configured to estimate a blood flow velocity profile at the line within target blood vessel 540. FIG. 5B illustrates a graphical representation of automatic alignment 555 of a transversal array of ultrasonic transducers, according to some embodiments. During automatic alignment operation 555, ultrasonic transducer array 570 is placed on a human body transversally to target blood vessel 565. Ultrasonic transducer array 570 is a linear array of ultrasonic transducers, of which ultrasonic transducer 575 is a representative ultrasonic transducer.

A plurality of instances of an ultrasonic scanning operation directed towards target blood vessel 565 using ultrasonic transducer array 570 is performed in a scanning direction, as indicated in FIG. 5B. In some embodiments, each ultrasonic transducer is activated independently during an instance of the ultrasonic scanning operation. In other embodiments, subsets of ultrasonic transducers are activated during an instance of the ultrasonic scanning operation (e.g., using beamforming). An instance of the ultrasonic scanning operation generates an ultrasonic signal toward target blood vessel 565 and receives at least one reflected ultrasonic signal from target blood vessel 565 at a receiving ultrasonic transducer. In accordance with various embodiments, a signal amplitude and/or a time of flight are determined for each of the at least one reflected ultrasonic signal of each instance of the ultrasonic scanning operation. In other embodiments, other properties or characteristics are determined, such as a phase shift or a Doppler signal. The signal amplitude and/or a time of flight are analyzed to determine which ultrasonic scanning operation, and thus which receiving ultrasonic transducer, exhibits alignment with target blood vessel 565.

As illustrated in FIG. 5B, ultrasonic transducer 580 of ultrasonic transducer array 570 is the ultrasonic transducer exhibiting alignment with target blood vessel 565. As shown, ultrasonic transducer 580 of ultrasonic transducer array 570 is most closely aligned with the center of target blood vessel 565. It should be appreciated that automatic alignment operation 555 can be applied to an ultrasonic sensing device including more than one linear array of ultrasonic transducers.

It should be appreciated that an ultrasonic sensor performing the described automatic alignment can be placed in any transversal location relative to the underlying target blood vessel other than exactly parallel to the target blood vessel. It should be appreciated that FIG. 5B is a generalization of an automatic alignment operation so as to illustrate the described embodiments without obfuscation, and that in practice a blood vessel might have variations in size and shape. In general, the automatic alignment operation described herein is operable so long as the target blood vessel is in the field of view of ultrasonic transducer array and is positioned transversally to the target blood vessel.

In some embodiments, the processing unit is configured to generate a Color Flow Doppler (CFD) line of the blood flow velocity within the blood vessel using the Doppler angle derived at least in part on using triangulation. In some embodiments, the processing unit is configured to calculate blood flow volume from the estimate of the blood flow velocity profile at the line within target blood vessel 540. In some embodiments, the processing unit is configured to determine a lumen area based at least in part on using the reflected ultrasonic signals received at linear arrays 520 and 530 to determine the wall position of target blood vessel 540 at the line over progressive instances of the reflected ultrasonic signals and extrapolating to determine the lumen area. In some embodiments, the processing unit is configured to determine vessel wall position using at least an imaging mode to extract blood flow velocity over an outer region of the lumen area using interpolation and over progressive instances of the reflected ultrasonic signals.

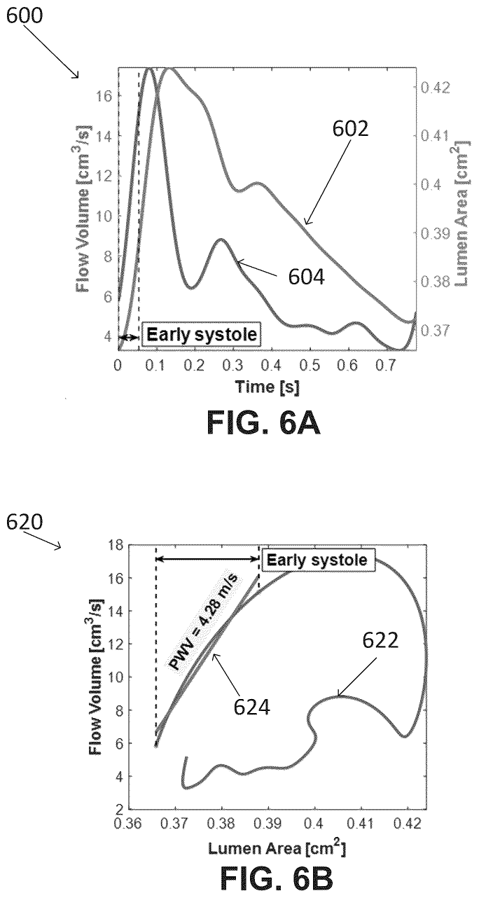

FIGS. 6A and 6B illustrate different graphs of a cardiac cycle, according to embodiments. FIG. 6A illustrates graph 600 of flow volume over time (line 604) and lumen area over time (line 602), according to embodiments. The ultrasonic sensing system of the described embodiments allows for tracking of blood vessel (e.g., arterial) wall motion over the cardiac cycle with high spatial resolution by using the variation of the phase of the echo signal at the wall position. In some embodiments, the lumen of the blood vessel is fitted with a circle passing at near and far wall positions. The instantaneous variation of the diameter is then evaluated using the phase and the lumen area is derived for each frame. This enables robust and time-resolved estimates of the lumen area, as shown in graph 600 of FIG. 6A. It should be appreciated that other automatic algorithms can also be applied to calculate this quantity.

FIG. 6B illustrates graph 620 of blood flow volume over lumen area, according to embodiments. Having an accurate measurement of the BFV and the lumen area over the cardiac cycle it is possible to extract the local PWV. The propagation of pressure (P) and flow (Q) waves in the arterial system is governed by the wave equation, with pulse wave velocity is shown in Equation 7:

$$P W V = \sqrt{\frac{\overline{A}}{ρ} \frac{1}{{C}_{A}}} \qquad ( 7 )$$

where Ā is the time-averaged cross-sectional area of the vessel, ρ the blood density, and C_{A}=dA/dP the local area compliance of the vessel. Another quantity relating pressure and flow waves in the arterial system is the characteristic impedance which, in absence of reflections, is defined as Zc=dP/dQ. The characteristic impedance is hence related to compliance, area and flow is shown in Equation 8:

$$Z c = \frac{d P}{d A} \frac{d A}{d Q} \qquad ( 8 )$$

That can be also written as Equation 9:

$$P W {V}_{Q A} = \frac{dQ}{dA} \qquad ( 9 )$$

Therefore, PWV can be estimated as the ratio between the change in flow and change in cross-sectional area during the reflection-free period of the cardiac cycle, e.g., the early systole. Line 622 of graph 600 represents the flow versus cross-sectional area graph in time. The PW is computed as the slope of line 624 overlaid to the loop, e.g., the linear fitting in the early systole time interval.

For a given volume of blood flow, if the cross-sectional area of the blood vessel is large, the velocity of the blood (and thus the pulse wave) will be lower. Conversely, if the blood vessel is narrower, the velocity will be higher. However, as the blood vessel becomes stiffer (less elastic), the PW increases, regardless of the cross-sectional area. It's important to note that the reflected wave from bifurcations or changes in blood vessel stiffness can affect the blood flow and pressure, leading to changes in PWV. Understanding these relationships helps in assessing cardiovascular health and the effects of diseases that alter arterial properties, such as atherosclerosis or hypertension.

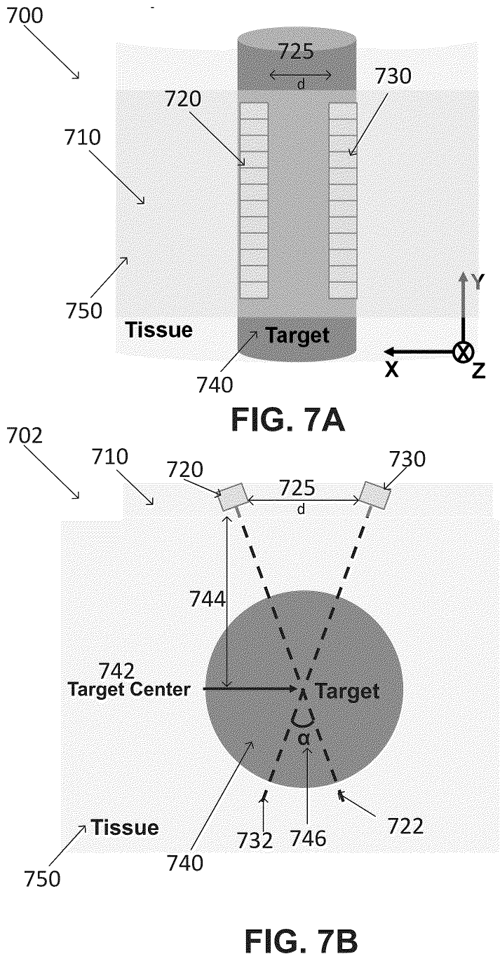

FIGS. 7A and 7B illustrate different views of a graphical representation of an ultrasonic sensing system 710 including two linear arrays 720 and 730 of ultrasonic transducers for performing cardiovascular monitoring positioned longitudinal to target blood vessel 740, according to embodiments. The illustrated embodiment positions linear arrays 720 and 730 parallel to target blood vessel 740. This allows for the extraction of the probe-to-vessel angle (by analyzing longitudinal B-mode images) and the blood velocity information on one plane. In some embodiments, this assumes that the flow is symmetric with respect to its axis to assess the blood flow volume quantitatively. For example, this configuration would be useful to evaluate turbulent flow near a stenosis or a bifurcation.

FIG. 7A illustrates a top view 700 of the ultrasonic sensing system 710. Linear arrays 720 and 730 are in parallel in a longitudinal direction at a fixed separation distance (d) 725 from each other. Ultrasonic sensing system 710 allows for the transmission of ultrasonic signals between ultrasonic transducers of the linear arrays 720 and 730 and tissue 750 in contact with ultrasonic sensing system 710 for sensing target blood vessel 740.

FIG. 7B illustrates a view 702 of ultrasonic sensing system 710. Ultrasonic signals 722 are transmitted from linear array 720 and ultrasonic signals 732 are transmitted from linear array 730 when ultrasonic sensing system 710 is in contact with tissue 750. Linear array 720 and linear array 730 are separated at fixed separation distance 725, and have a fixed relative angle (α) 746, such that ultrasonic signals 722 and 732 intersect at intersection line 742, which is at fixed transmission distance 744 from ultrasonic sensing system 710. Linear array 720 and linear array 730 each image target blood vessel 740 on a different cross-sectional plane and the two planes intersect at intersection line 742 that is at fixed transmission distance 744 from the sensor surface. Fixed transmission distance (zᵢ) 744 is determined as shown in Equation 1 above. Ultrasonic sensing system 710 is configured to have the center of the target blood vessel 740 on or in close proximity to intersection line 742.

In some embodiments, the ultrasonic sensing system further includes a third linear array of ultrasonic transducers coupled to the substrate, the third linear array of ultrasonic transducers for transmitting ultrasonic signals that intersect with the ultrasonic signals transmitted from the second linear array of ultrasonic signals transmitted in the second direction. The third linear array of ultrasonic transducers is fixedly positioned in parallel in the longitudinal direction to the first linear array of ultrasonic transducers and the second linear array of ultrasonic transducers and at a second fixed separation distance from the second linear of ultrasonic transducers, and wherein the third linear array of ultrasonic transducers and the second linear array of ultrasonic transducers are positioned such that the ultrasonic signals transmitted from the third linear array intersect with the ultrasonic signals transmitted from the second linear array in the second direction at a second fixed angle and intersect at a second fixed transmission distance. In some embodiments, the third linear array of ultrasonic transducers is positioned for transmitting ultrasonic signals in the first direction, wherein the second fixed angle is equal to the fixed angle, and wherein the third linear array of ultrasonic transducers and the second linear array of ultrasonic transducers are positioned such that the ultrasonic signals transmitted from the third linear array in the first direction intersect with the ultrasonic signals transmitted from the second linear array at the second fixed transmission distance.

In some embodiments, the ultrasonic sensing system further includes a fourth linear array of ultrasonic transducers coupled to the substrate, the fourth linear array of ultrasonic transducers for transmitting ultrasonic signals that intersect with the ultrasonic signals transmitted from the first linear array of ultrasonic signals transmitted in the first direction. The fourth linear array of ultrasonic transducers is fixedly positioned in parallel in the longitudinal direction to the first linear array of ultrasonic transducers and the second linear array of ultrasonic transducers and at a third fixed separation distance from the first linear of ultrasonic transducers, and wherein the fourth linear array of ultrasonic transducers and the first linear array of ultrasonic transducers are positioned such that the ultrasonic signals transmitted from the fourth linear array intersect with the ultrasonic signals transmitted from the first linear array in the first direction at a third fixed angle and intersect at a third fixed transmission distance. In some embodiments, the fourth linear array of ultrasonic transducers is positioned for transmitting ultrasonic signals in the second direction, wherein the third fixed angle is equal to the fixed angle, and wherein the fourth linear array of ultrasonic transducers and the first linear array of ultrasonic transducers are positioned such that the ultrasonic signals transmitted from the fourth linear array in the second direction intersect with the ultrasonic signals transmitted from the first linear array at the third fixed transmission distance.

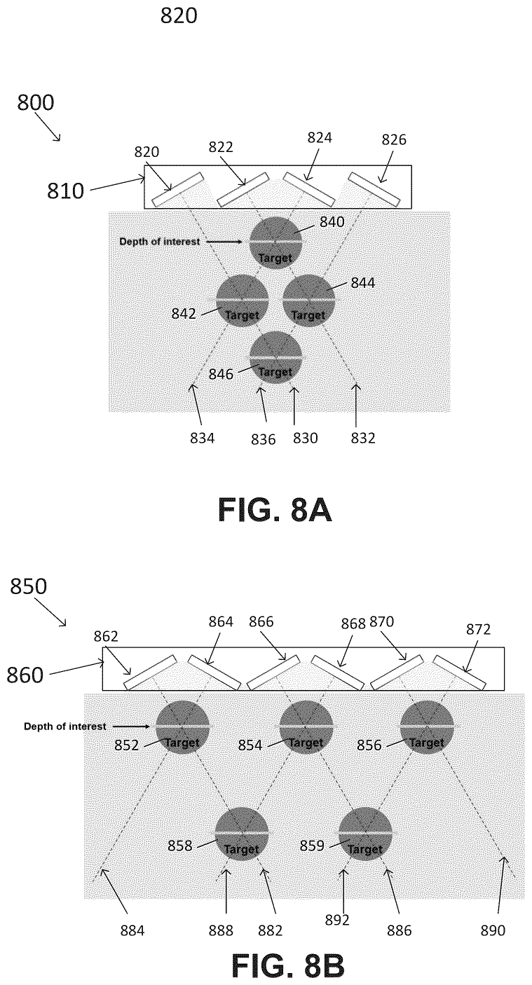

FIGS. 8A and 8B illustrate cross-sectional views of ultrasonic sensing systems including more than two linear arrays of ultrasonic transducers, according to some embodiments. Adding additional arrays of ultrasonic transducers to an ultrasonic sensing system allows for the sensing of planes intersecting at different depths and positions, depending on the selected pairs of arrays, increasing the flexibility to image more targets or make the system more robust against the target location. This can be obtained by adding additional arrays with similar orientation as illustrated in FIG. 8A or with alternating orientations as illustrated in FIG. 8B.

FIG. 8A illustrates a cross-sectional view 800 of ultrasonic sensing system 810 comprising ultrasonic transducer arrays 820, 822, 824, and 826. Ultrasonic transducer arrays 820, 822, 824, and 826 are parallel in a longitudinal direction at fixed separation distances from each other. Ultrasonic transducer arrays 820, 822, 824, and 826 are angled relative to each other such that ultrasonic signals 830 and 832 transmitted from ultrasonic transducer arrays 820 and 822, respectively, intersect with ultrasonic signals 834 and 836 transmitted from ultrasonic transducer arrays 824 and 826. In some embodiments, the ultrasonic signals 830, 832, 834, and 836 are transmitted at fixed angles relative to each other and intersect at fixed transmission distances. It should be appreciated that in such embodiments, during operation ultrasonic transducer arrays 820, 822, 824, and 826 are fixedly positioned such that the fixed angles and fixed transmission distances are known.

As illustrated, ultrasonic signal 830 intersects ultrasonic signal 834 at target 842 and intersects ultrasonic signal 836 at target 846, and ultrasonic signal 832 intersects ultrasonic signal 834 at target 840 and intersects ultrasonic signal 836 at target 844. As illustrated, ultrasonic transducer arrays 820, 822, 824, and 826 are shown in parallel with target blood vessels 840, 842, 844, and 846. It should be appreciated that ultrasonic transducer arrays 820, 822, 824, and 826 of ultrasonic sensing system can be placed in any position relative to target blood vessels 840, 842, 844, and 846, such as transversally, or longitudinally depending on the information that needs to be extracted and with some angular tolerance.

FIG. 8B illustrates a cross-sectional view 850 of ultrasonic sensing system 860 comprising ultrasonic transducer arrays 862, 864,866, 868, 870, and 872. Ultrasonic transducer arrays 862, 864, 866, 868, 870, and 872 are parallel in a longitudinal direction at fixed separation distances from each other. Ultrasonic transducer arrays 862, 864, 866, 868, 870, and 872 are angled relative to each other such that ultrasonic signals 882, 886, and 890 transmitted from ultrasonic transducer arrays 862, 866, and 870, respectively, intersect with ultrasonic signals 884, 888, and 892 transmitted from ultrasonic transducer arrays 864, 868, and 872. In some embodiments, the ultrasonic signals 862, 864, 866, 868, 870, and 872 are transmitted at fixed angles relative to each other and intersect at fixed transmission distances. It should be appreciated that in such embodiments, during operation ultrasonic transducer arrays 862, 864,866, 868, 870, and 872 are fixedly positioned such that the fixed angles and fixed transmission distances are known.

As illustrated, ultrasonic signal 882 intersects ultrasonic signal 884 at target 852 and intersects ultrasonic signal 888 at target 858, ultrasonic signal 886 intersects ultrasonic signal 888 at target 854 and intersects ultrasonic signal 892 at target 859, and ultrasonic signal intersects 890 intersects ultrasonic signal 892 at target 856. As illustrated, ultrasonic transducer arrays 862, 864, 866, 868, 870, and 872 are shown in parallel with target blood vessels 852, 854, 856, 858, and 859. It should be appreciated that ultrasonic transducer arrays 862, 864, 866, 868, 870, and 872 of ultrasonic sensing system can be placed in any position relative to target blood vessels 852, 854, 856, 858, and 859, such as transversally or longitudinally depending on the information that needs to be extracted and with some angular tolerance.

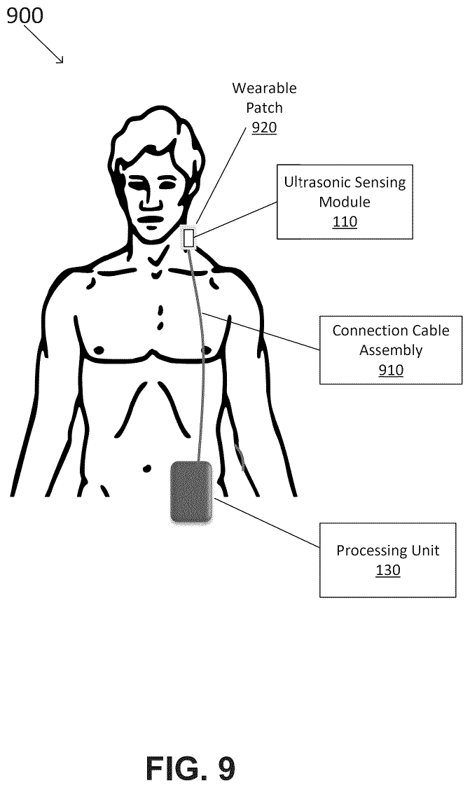

FIG. 9 illustrates placement 900 of the components of a wearable system for cardiovascular monitoring, where the wearable system is a wearable patch 920 including ultrasonic sensing module 110. As illustrated, wearable patch 920 is placed on the neck of a user such that ultrasonic sensing module 110 is proximate the common carotid artery, and is held in place using, or comprised within, a flexible adhesive patch. In placement 900, processing unit 130 is worn on the lower abdomen of the user, and held in place using an adhesive patch, a strap, an elastic band, or any other attachment means. Connection cable assembly 910 runs along the abdomen and chest and is coupled to ultrasonic sensing module 110 near the carotid artery and processing unit 130. It should be appreciated that connection cable assemblies 910 may be held in place by an attachment means, such as an adhesive, a strap, an elastic band, etc. It should be appreciated that wearable patch 920 can be placed anywhere on the human body proximate a blood vessel for performing cardiovascular monitoring.

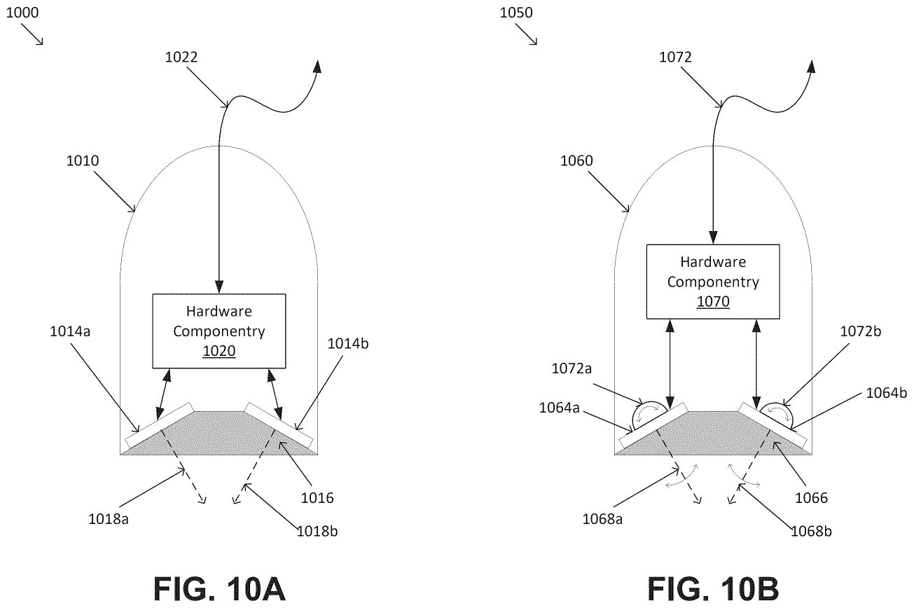

In some embodiments, the ultrasonic sensing system further includes a probe housing configured to be manually positioned on a body proximate a blood vessel. FIGS. 10A and 10B illustrate graphical representations of probe housings including an ultrasonic sensor module, according to some embodiments. FIG. 10A illustrates ultrasonic sensing system 1000 including probe housing 1010 comprising linear array 1014a, linear array 1014b, and hardware componentry 1020 communicatively coupled to a processing unit (e.g., processing unit 130). Probe housing 1010 is a handheld device for manual operation by a human user (e.g., an ultrasound technician or a patient) for placement on a body for performing ultrasonic sensing into the tissue for performing cardiovascular monitoring. It should be appreciated that probe housing 1010 can have any form factor for allowing the human user to manually manipulate probe housing 1010. For example, probe housing 1010 may be ergonomically designed, may include gripping materials on the exterior (e.g., rubber, ribbing, etc.)

Linear arrays 1014a and 1014b are configured to transmit ultrasonic signals 1018a and 1018b, respectively, through substrate 1016 (e.g., an acoustic coupling substrate) into human tissue, such that ultrasonic signals 1018a and 1018b intersect at an intersection point at the fixed transmission distance within a target blood vessel. It should be appreciated that hardware componentry 1020 and linear arrays 1014a and 1014b operate in a similar manner as described above in accordance with ultrasonic sensor module 110 of FIGS. 1 through 3B and ultrasonic sensing system 410 of FIG. 4. Hardware componentry 1020 is configured to communicate reflected ultrasonic signals to a processing unit (e.g., processing unit 130) over connection cable 1022.

In accordance with some embodiments, the fixed angle of intersection can be controlled during transmission to control a depth at which ultrasonic sensing is performed in the tissue. FIG. 10B illustrates ultrasonic sensing system 1050 including probe housing 1060 comprising linear array 1064a, linear array 1064b, and hardware componentry 1020 communicatively coupled to a processing unit (e.g., processing unit 130). Probe housing 1060 is a handheld device for manual operation by a human user (e.g., an ultrasound technician or a patient) for placement on a body for performing ultrasonic sensing into the tissue for performing cardiovascular monitoring. It should be appreciated that probe housing 1060 can have any form factor for allowing the human user to manually manipulate probe housing 1060. For example, probe housing 1060 may be ergonomically designed, may include gripping materials on the exterior (e.g., rubber, ribbing, etc.)

Linear arrays 1064a and 1064b are configured to transmit ultrasonic signals 1068a and 1068b, respectively, through substrate 1066 (e.g., an acoustic coupling substrate) into human tissue, such that ultrasonic signals 1068a and 1068b intersect at an intersection point at the fixed transmission distance within a target blood vessel. Mechanical control mechanisms 1072a and 1072b are coupled to linear arrays 1064a and 1064b, respectively, for controlling an angle at which ultrasonic signals 1068a and 1068b are transmitted into the human tissue, such that the depth of the point of intersection of ultrasonic signals 1068a and 1068b is controlled. In some embodiments, changes are made to the projection angles of linear arrays 1064a and 1064b before or between transmission operations. As such, the fixed angle of intersection and the distance of the point of intersection of ultrasonic signals 1068a and 1068b from ultrasonic sensing system 1050 is known during operation. It should be appreciated that hardware componentry 1070 and linear arrays 1064a and 1064b operate in a similar manner as described above in accordance with ultrasonic sensor module 110 of FIGS. 1 through 3B and ultrasonic sensing system 410 of FIG. 4. Hardware componentry 1070 is configured to communicate reflected ultrasonic signals to a processing unit (e.g., processing unit 130) over connection cable 1072.

### Conclusion

The examples set forth herein were presented in order to best explain, to describe particular applications, and to thereby enable those skilled in the art to make and use embodiments of the described examples. However, those skilled in the art will recognize that the foregoing description and examples have been presented for the purposes of illustration and example only. Many aspects of the different example embodiments that are described above can be combined into new embodiments. The description as set forth is not intended to be exhaustive or to limit the embodiments to the precise form disclosed. Rather, the specific features and acts described above are disclosed as example forms of implementing the claims.

Reference throughout this document to “one embodiment,” “certain embodiments,” “an embodiment,” “various embodiments,” “some embodiments,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics of any embodiment may be combined in any suitable manner with one or more other features, structures, or characteristics of one or more other embodiments without limitation.

What is claimed is:

1. An ultrasonic sensing system comprising:
a substrate;
a first linear array of ultrasonic transducers coupled to the substrate, the first linear array of ultrasonic transducers for transmitting ultrasonic signals in a first direction;
a second linear array of ultrasonic transducers coupled to the substrate, the second linear array of ultrasonic transducers for transmitting ultrasonic signals in a second direction; and
hardware componentry for controlling transmission of the ultrasonic signals at the first linear array of ultrasonic transducers and the second linear array of ultrasonic transducers and for controlling receipt of reflected ultrasonic signals at the first linear array of ultrasonic transducers and the second linear array of ultrasonic transducers;
wherein the first linear array of ultrasonic transducers and the second linear array of ultrasonic transducers are fixedly positioned in parallel in a longitudinal direction at a fixed separation distance, and wherein the first linear array of ultrasonic transducers and the second linear array of ultrasonic transducers are positioned such that the ultrasonic signals transmitted in the first direction intersect with the ultrasonic signals transmitted in the second direction at a fixed angle and intersect at a fixed transmission distance.

2. The ultrasonic sensing system of claim 1, further comprising:
a wearable patch configured to be attached to a body proximate a blood vessel, wherein the substrate, the first linear array of ultrasonic transducers and the second linear array of ultrasonic transducers are coupled to the wearable patch and positioned such that the first linear array of ultrasonic transducers and the second linear array of ultrasonic transducers can project ultrasonic signals into the body.

3. The ultrasonic sensing system of claim 1, further comprising:
a probe housing configured to be manually positioned on a body proximate a blood vessel, wherein the substrate, the first linear array of ultrasonic transducers and the second linear array of ultrasonic transducers are coupled to the probe housing and positioned such that the first linear array of ultrasonic transducers and the second linear array of ultrasonic transducers can project ultrasonic signals into the body.

4. The ultrasonic sensing system of claim 3, further comprising:
a mechanical control mechanism disposed within the probe housing and movably coupled to the first linear array of ultrasonic transducers and the second linear array of ultrasonic transducers, the mechanical control mechanism configured to control the fixed angle of the ultrasonic signals transmitted in the first direction and the ultrasonic signals transmitted in the second direction.

5. The ultrasonic sensing system of claim 1, further comprising:
a third linear array of ultrasonic transducers coupled to the substrate, the third linear array of ultrasonic transducers for transmitting ultrasonic signals that intersect with the ultrasonic signals transmitted from the second linear array of ultrasonic signals transmitted in the second direction;
wherein the third linear array of ultrasonic transducers is fixedly positioned in parallel in the longitudinal direction to the first linear array of ultrasonic transducers and the second linear array of ultrasonic transducers and at a second fixed separation distance from the second linear of ultrasonic transducers, and wherein the third linear array of ultrasonic transducers and the second linear array of ultrasonic transducers are positioned such that the ultrasonic signals transmitted from the third linear array intersect with the ultrasonic signals transmitted from the second linear array in the second direction at a second fixed angle and intersect at a second fixed transmission distance.

6. The ultrasonic sensing system of claim 5, wherein the third linear array of ultrasonic transducers is positioned for transmitting ultrasonic signals in the first direction, wherein the second fixed angle is equal to the fixed angle, and wherein the third linear array of ultrasonic transducers and the second linear array of ultrasonic transducers are positioned such that the ultrasonic signals transmitted from the third linear array in the first direction intersect with the ultrasonic signals transmitted from the second linear array at the second fixed transmission distance.

7. The ultrasonic sensing system of claim 5, further comprising:
a fourth linear array of ultrasonic transducers coupled to the substrate, the fourth linear array of ultrasonic transducers for transmitting ultrasonic signals that intersect with the ultrasonic signals transmitted from the first linear array of ultrasonic signals transmitted in the first direction;
wherein the fourth linear array of ultrasonic transducers is fixedly positioned in parallel in the longitudinal direction to the first linear array of ultrasonic transducers and the second linear array of ultrasonic transducers and at a third fixed separation distance from the first linear of ultrasonic transducers, and wherein the fourth linear array of ultrasonic transducers and the first linear array of ultrasonic transducers are positioned such that the ultrasonic signals transmitted from the fourth linear array intersect with the ultrasonic signals transmitted from the first linear array in the first direction at a third fixed angle and intersect at a third fixed transmission distance.

8. The ultrasonic sensing system of claim 7, wherein the fourth linear array of ultrasonic transducers is positioned for transmitting ultrasonic signals in the second direction, wherein the third fixed angle is equal to the fixed angle, and wherein the fourth linear array of ultrasonic transducers and the first linear array of ultrasonic transducers are positioned such that the ultrasonic signals transmitted from the fourth linear array in the second direction intersect with the ultrasonic signals transmitted from the first linear array at the third fixed transmission distance.

9. The ultrasonic sensing system of claim 1 further comprising:
a processing unit for processing the reflected ultrasonic signals received at the first linear array of ultrasonic transducers and the second linear array of ultrasonic transducers for performing cardiovascular monitoring.

10. The ultrasonic sensing system of claim 9, wherein the first linear array of ultrasonic transducers and second linear array of ultrasonic transducers are configured to transmit ultrasonic signals into a blood vessel such that the ultrasonic signals intersect at an intersection point at the fixed transmission distance within the blood vessel; and
wherein the processing unit is configured to determine mean phase changes of consecutive instances of the reflected ultrasonic signals received at the first linear array of ultrasonic signals and the second linear array of ultrasonic signals, is configured to determine blood flow velocity at the intersection point based at least in part on the mean phase changes, and is configured to determine blood flow direction and Doppler angle at the intersection point by using triangulation to extract blood velocity components from the mean phase changes of consecutive instances of the reflected ultrasonic signals.

11. The ultrasonic sensing system of claim 10, wherein the processing unit is configured to generate a Color Flow Doppler (CFD) image of the blood flow velocity within the blood vessel using the Doppler angle derived at least in part on using triangulation.

12. The ultrasonic sensing system of claim 11, wherein the processing unit is configured to calculate blood flow volume from the blood flow velocity within a lumen area of the blood vessel.

13. The ultrasonic sensing system of claim 12, wherein the processing unit is configured to determine the lumen area based at least in part on using the reflected ultrasonic signals received at the first linear array of ultrasonic transducers and the second linear array of ultrasonic transducers to determine wall position of the blood vessel over progressive instances of the reflected ultrasonic signals.

14. The ultrasonic sensing system of claim 13, wherein the processing unit is configured to determine vessel wall position using at least an imaging mode to extract the blood flow velocity over an outer region of the lumen area using interpolation and over progressive instances of the reflected ultrasonic signals.

15. The ultrasonic sensing system of claim 13, wherein the processing unit is configured to determine a pulse wave velocity within the blood vessel based at least in part on the blood flow velocity and the lumen area.

16. The ultrasonic sensing system of claim 9, wherein the processing unit is configured to determine a lumen area based at least in part on using the reflected ultrasonic signals received at the first linear array of ultrasonic transducers and the second linear array of ultrasonic transducers to determine wall position of a blood vessel.

17. The ultrasonic sensing system of claim 9, wherein the processing unit is configured to perform an imaging mode to obtain morphological images of a blood vessel based at least in part on the reflected ultrasonic signals received at the first linear array of ultrasonic transducers and the second linear array of ultrasonic transducers.

18. The ultrasonic sensing system of claim 10, wherein the processing unit is configured to perform alignment of the ultrasonic signals transmitted by the first linear array of ultrasonic transducers and second linear array of ultrasonic transducers to identify a line aligned with a center of a blood vessel, and configured to estimate a blood flow velocity profile at the line within the blood vessel.

19. The ultrasonic sensing system of claim 18, wherein the processing unit is configured to generate a Color Flow Doppler (CFD) of the line of the blood flow velocity within the blood vessel using the Doppler angle derived at least in part on using triangulation.

20. The ultrasonic sensing system of claim 19, wherein the processing unit is configured to calculate blood flow volume from the estimate of the blood flow velocity profile at the line within the blood vessel.

21. The ultrasonic sensing system of claim 20, wherein the processing unit is configured to determine a lumen area based at least in part on using the reflected ultrasonic signals received at the first linear array of ultrasonic transducers and the second linear array of ultrasonic transducers to determine wall position of the blood vessel at the line over progressive instances of the reflected ultrasonic signals and extrapolating to determine the lumen area.

22. The ultrasonic sensing system of claim 21, wherein the processing unit is configured to determine vessel wall position using at least an imaging mode to extract blood flow velocity over an outer region of the lumen area using interpolation and over progressive instances of the reflected ultrasonic signals.
